(19) United States
(12) Patent Application Publication
(10) Pub. No.: US 2006/0079910 A1
(43) Pub. Date: Apr. 13, 2006

(54) Surgical marker

(76) Inventor: Arthur A. Tartaglia, Richmond Heights, OH (US)

Correspondence Address: Jonathan A. Withrow;Fay, Sharpe, Fagan, Minnich & McKee, LLP, Seventh Floor, 1100 Superior Avenue, Cleveland, OH, 44114-2579, US

(21) Appl. No.: 11/282,856

(22) Filed: Nov. 17, 2005

Related U.S. Application Data
(63) Continuation-in-part of application No. 10/657,889, filed on Sep. 9, 2003, now abandoned.
(60) Provisional application No. 60/409,395, filed on Sep. 10, 2002.

(51) Int. Cl. A61B 17/00

(52) U.S. Cl. 606/116

(57) ABSTRACT

A marking pen for indicating the proper location of incisions to be made on a patient undergoing surgery is provided. The marking pen includes a handle having a first end and a second end; a wheel comprising a gelatinous ink-soaked polymer rotably attached to the first end of the handle; and a plurality of treads on a periphery of the wheel.

This application is a continuation-in-part of U.S. patent application Ser. No. 10/657,889 filed Sep. 9, 2003, which claims priority to U.S. Provisional Application Ser. No. 60/409,395 filed Sep. 10, 2002, each which is incorporated by reference.

### BACKGROUND OF THE INVENTION

The present invention relates, in general, to a marking pen, more particularly, to a marking pen for use during surgery, so as to guide the surgeon in making a proper incision. The pen may also aid the surgeon in marking where to place sutures, staples, glue or other mechanisms for closing an incision post operatively.

The use of marking pens in surgery is common. A surgeon will mark lines on a patient's body so as to know the proper place and length of the incision that will be made during the operation. Such lines can be particularly important in plastic surgery. In such cases, it is extremely important that the incisions be at the proper location, and of the appropriate length. This is often difficult to do during surgery without the aid of markings.

Typically, a surgeon will use a felt tip pen to mark lines representing the desired incisions. Felt tip pens can drag across the patient's skin catching some of the skin. The skin that is caught by the pen can bunch up underneath the pen resulting in a jagged or crooked line. Also, felt tip pens, in particular, can “bleed” when they are first placed on a patient's skin.

Marking pens of the prior art also have a tendency to dry out. Prior art pens have tried to solve this problem of drying out by providing a surgical marker with a reservoir that feeds a marking portion of the surgical marker. In these pens having reservoirs, the channels leading to the marking portion can become clogged.

Due to these problems with marking pens, some surgeons rely on other marking techniques. Some doctors dip toothpicks or other similar instruments in ink, and then drag the ink-soaked toothpick across the patient's skin to form a line. The toothpicks, or other similar marking instruments do not hold a significant amount of ink and must be repeatedly dipped into the ink when the surgeon is drawing a number of lines.

Accordingly, to overcome these problems, there is a need for a marking pen that enables a surgeon to make a line on the patient's skin that is not jagged. Additionally, the line can provide configurations that can mark a patient's skin to aid the doctor in the placement of sutures after the surgery has been performed.

### SUMMARY OF THE INVENTION

A method of marking a patient's skin for surgery includes marking a patient's skin with ink using a surgical marker without penetrating the patient's skin. The surgical marker includes a handle and a wheel rotatively attached to the handle. The wheel includes a marking surface for the depositing ink onto the patient's skin.

A surgical marker kit includes a sterile handle, a sterile wheel, ink, and a sealed ink container. The handle includes a first end portion and a second end portion. The wheel is configured to attach to the first end portion of the handle. The wheel includes a circumferential marking surface. The wheel is made of a material that selectively retains and dispenses ink. The sealed ink container includes a selectively removable portion to provide access to the ink.

A surgical marker includes a handle and a wheel rotatively attached to the handle. The wheel includes a first end portion and a second end portion. The wheel rotatively attaches to the first end portion of the handle. The wheel includes a flat or arcuate marking surface disposed at a peripheral edge of the wheel.

### BRIEF DESCRIPTION OF THE DRAWINGS

FIG. 1 is an exploded view of a surgical marker.

FIG. 2 is an exploded view of the surgical marker, opposite the view shown in FIG. 1.

FIG. 3 is a perspective view of an alternative embodiment of a surgical marker.

FIG. 4 is a perspective view of another embodiment of a surgical marker.

FIG. 5 is a perspective view of a first end portion of the surgical marker of FIG. 3 showing a first embodiment of a marking wheel attached to the first end portion.

FIG. 6 is a perspective view of the first end portion of the surgical marker of FIG. 3 showing a second embodiment of a marking wheel attached to the first end portion.

FIG. 7 is a drawing of an example of a surgical marker kit.

### DETAILED DESCRIPTION OF THE INVENTION

It is to be understood that the specific devices and processes illustrated in the attached drawings, and described in the following specification are simply exemplary embodiments of the inventive concepts. Therefore, specific examples and characteristics relating to the embodiments disclosed herein are not to be considered as limiting.

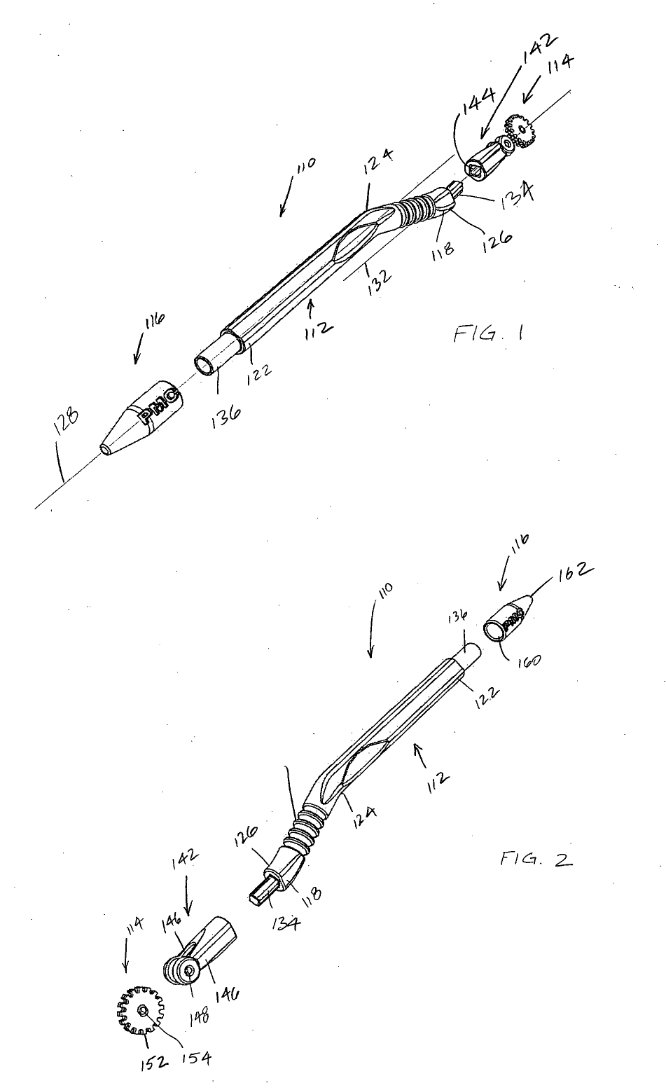

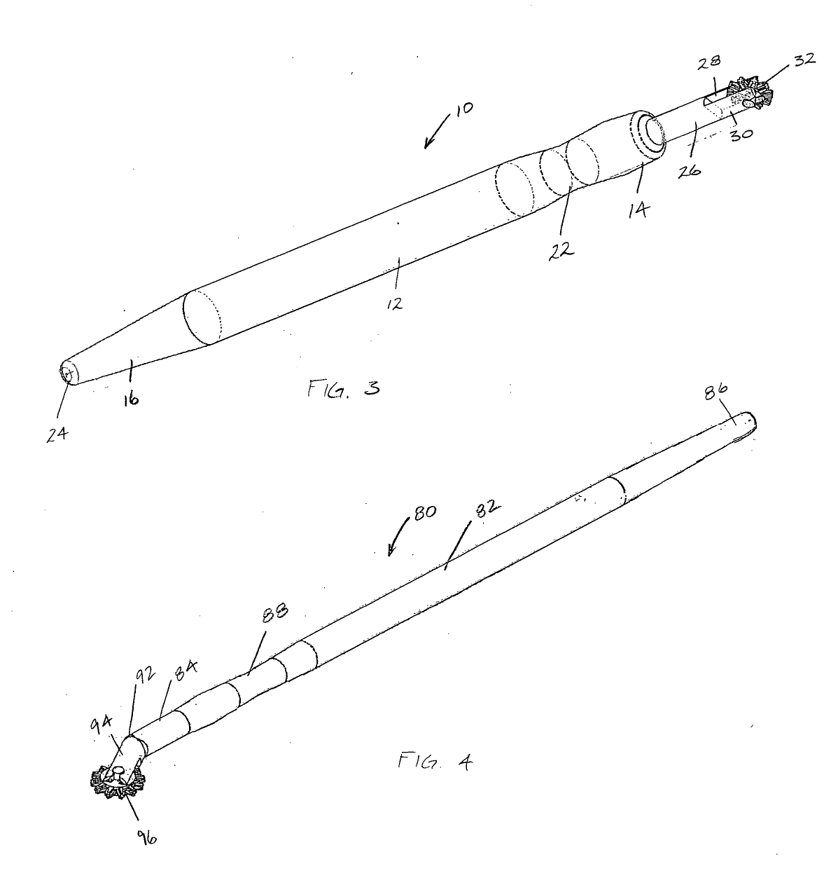

With reference to FIG. 1, a surgical marker 110 generally includes a handle 112, a wheel 114 that attaches to the handle and a nib 116 that also attaches to the handle. The handle 112 in the embodiment depicted in FIGS. 1 and 2 can be made of any conventional material, such as plastic or stainless steal. Also, the handle 110 can be made of material that can withstand sterilization via gamma radiation. The handle, which can also be referred to as the body, need not include a reservoir for the ink that is eventually deposited on a patient's skin. The handle 112 includes a first end portion 118 and a second end portion 122. The handle 110 includes two bends: a first bend 124 and a second bend 126. The second bend 126 is located nearer the first end portion of 118 of the handle 112 as compared to the first bend 124. The portion of the handle 110 rearward of the first bend 124, i.e. towards the second end portion 122, is aligned generally along a first axis 128. The portion of the handle 110 forward of the second bend 126 is aligned generally along a second axis 132 that is parallel with the first axis 128.

The handle further includes a wheel mount extension 134 that extends forwardly from the first end portion 118 of the handle 110 generally coaxial with the second axis 132. As more clearly seen FIG. 2, the wheel mount extension 134 is polygonal in a cross section taken normal to the second axis 132. Such a polygonal configuration provides multiple predetermined orientations at which the wheel 114 can attach the handle 112. The handle 110 also includes a nib extension 136 that extends generally rearwardly from the handle 112 aligned along and coaxial with the first axis 128. In the depicted embodiment, the nib mounting extension 136 is generally circular in a cross section taken normal to the first axis 128. Either mounting extension can take alternative configurations.

Annular ridges 138 are also provided on the handle 112 between the first bend 124 and the second bend 126. This location is typically where the marking pen is gripped during use. The annular ridges 138 provide for a cleaner gripping surface for the surgeon during the operation. The annular ridges 138 provide less surface area for contaminants to gather on the pen that can make gripping the pen difficult.

A wheel mount 142 attaches to the handle 112. In the depicted embodiment, the wheel mount 142 includes a polygonaly shaped opening 144 (FIG. 1) that receives the wheel mount extension 134. In alternative embodiment, the handle 112 can include the female portion of the connection between the handle and the wheel mount 142 and the wheel mount can include the male portion of the connection. In the depicted embodiment, the opening 144 is a polygonal shape that corresponds to the wheel mount extension 134 and provide for a number of predetermined mounting orientations in which the wheel 114 can mount to the handle 110. The orientations that the wheel mount 142 can take are fixed about the second axis 132, which is perpendicular to the axis of rotation for the wheel 114. The wheel mount 142 also includes tines 146 that are spaced from one another so that the wheel 114 is received between the tines. Openings 148 are provided in each tine 146. The openings 148 are aligned with one another so that an axle (not shown) is received in the openings to attach the wheel 114 to the wheel mount 142.

The wheel 114 in the depicted embodiment is similar and/or can be the same as the wheels that are described below for alternative embodiments of the surgical marker. The wheel 114 includes a plurality of treads 152 that define marking surfaces that will contact the patient's skin to provide a guideline for an incision that will be made during operation. The wheel also includes a central opening 154 through which the axle is received. The axle is also received in the opening 148 of the wheel mount 142 to attach the wheel 114 to the wheel mount. The wheel 114 in the depicted embodiment is made of an open cell material, for example, HDMP. To produce the wheel 114, powder material is placed into a form, the powder is then pressed and sintered. Other materials can also be used for the wheel. Since the wheel is not internally fed by a reservoir, the material from which the wheel is made should selectively retain ink to be deposited onto a patient's skin and selectively deposit that ink upon coming into contact with the skin. The embodiment depicted in FIGS. 1 and 2, the wheel 114 includes flat or arcuate marking surfaces that do not penetrate the patient's skin when marking the skin. Instead ink is deposited on the top layer of the patient's skin.

The marking nib 116 connects to the handle 112. The marking nib 116 in the depicted embodiment includes a circular opening 160 that is configured to receive the nib extension 136 that extends rearwardly from the second end portion 122 of the handle 112. Alternatively, the marking nib can include a male connection portion that cooperates with a female connection portion of the handle. In the depicted embodiment, the nib 116 can be made of the same or similar material as the wheel 114. Also, a reservoir can be provided in the handle 112. Ink can be provided in the reservoir and communicate with a marking end 162 of the nib 116. A surgeon can mark the starting and ending locations for an incision line using the pointed end 162 of the nib 116. The nib can either receive ink from a reservoir disposed in the handle 112 or the end 162 of the nib 116 can be deposited in an ink reservoir or contact an ink pad and selectively retain the ink until the ink is deposited on the patient's skin. The ink for both the wheel 114 and the nip 116 can either by retained by absorption or simply through surface tension that attracts the ink to the respective marking surfaces of the respective wheel 114 and the nip 116.

Referring to FIG. 3, another embodiment of a surgical marker 10 includes a body 12 having a first end portion 14 and a second end portion 18. The body 12 can make up at least a portion of a handle of the surgical marker. The body has a generally cylindrical shape and can be made of any conventional material, preferably plastic or stainless steel. Since this surgical marker does not require a reservoir, the body can be solid. A contoured portion 22 is located adjacent the first end portion 14. The contoured portion 22 can provide a more ergonomic handle for the surgical marker. The second end portion 18 has a tapered end 24 that tapers away from the central portion 16.

In the embodiment depicted in FIG. 3, a neck 26 attaches to the first end portion 14 of the body. The neck 26 can either be fastened to or an integral with the body 12. Where the neck 26 is integral with the body 12, the neck and the body are formed from a single piece. In the depicted embodiment, the neck is cylindrical having two arms, 28 and 30, that project away from the first end portion 14. A wheel 32 is rotatively attached between the arms 28, 30. The neck 26 and the arms 28, 30 can also be made from a solid piece of plastic, i.e. containing no channels inside. The neck 26 can detach from the body 12 so that the neck and wheel 32 can be discarded and the remainder of the marker can be re-used.

Referring now to FIG. 5, arm 28 includes two tines 34 and 36 and arm 30 includes two tines 38 and 40. Tines 34 and 36 and arm 28 define an arcuate notch 42 that receives an axle 44 of wheel 32 (FIG. 1). The arm 30 and tines 38 and 40 define an arcuate notch 46 that aligns with the arcuate notch 42. The arcuate notch 46 also receives the axle 44 of the wheel 32. The arcuate portions 42, 46 are adapted to surround more than half the periphery of the axle 42 of the wheel. Thus, the wheel can lock into the notch when mounted to the neck 26, yet the wheel can also be detached from the neck after use.

The wheel 32 includes a plurality of spaced treads 50. The wheel 32 can be made of a conventional material including an elastomer, a plastic, or other well known material. The wheel in one embodiment is made of a gelatinous ink-soaked polymer. More specifically the wheel comprises a plasticized (approximately 60% ink and approximately 40% resin) felt tip from the medical industry. Such polymers are available from Identity Group, 1480 Gould Drive, Cookeville, Tenn. and the Bacon Felt Co. Other ink-soaked resins could also be used including, but not limited to, polyethylene and polypropylene. The ink-soaked polymer is preferred because it reduces the tendency of the wheel to drag across the patient's skin, catching some of the skin and causing the skin to “bunch” together. Furthermore, the ink-soaked polymer wheel does not have a tendency to dry out. Also, the ink-soaked polymer wheel eliminates the need for an ink reservoir where channels leading from the reservoir to the wheel may clog. Also, a surgeon using such an ink-soaked polymeric wheel, when marking lines on the patient's skin, does not need to dip the surgical marker in ink prior to marking the patient's skin.

The marking agent or ink used with the wheel can be any conventional marking agent used to mark a patient's skin. An example of a marking agent is Gentian Violet, however other marking agents can be used.

The wheel 32 includes treads 50 having marking surfaces 52 disposed at a peripheral edge of each tread. The treads 50 are the shape of a frustum of a prism, however, the treads could take form in a number of different shapes. The marking surfaces 52 are spaced from one another resulting in a dotted line on the patient's skin when the surgeon makes the mark, however, the marking surfaces need not be spaced from one another when a continuous line is desired. The marking surfaces can be flat, or arcuate to match the radius of the wheel so that as large amount of marking surface as possible contacts the patient's skin. The marking surface can be square, circular or any other shape. The dots that are made by the wheel on the patient's skin can be spaced apart from one another the conventional distance at which post-operative sutures are placed, or some multiple thereof, i.e. every third dot a suture or other device to close the incision is placed.

The axle 44 can be separate from the wheel, or the wheel and axle can be made from one integral unit. As stated before, the axle is received in the arcuate notches 42, 46 of the arms 28, 30. The receipt of the axle in the notches allows the wheel to detach from the neck so that the remaining portion of the surgical marker can be re-used.

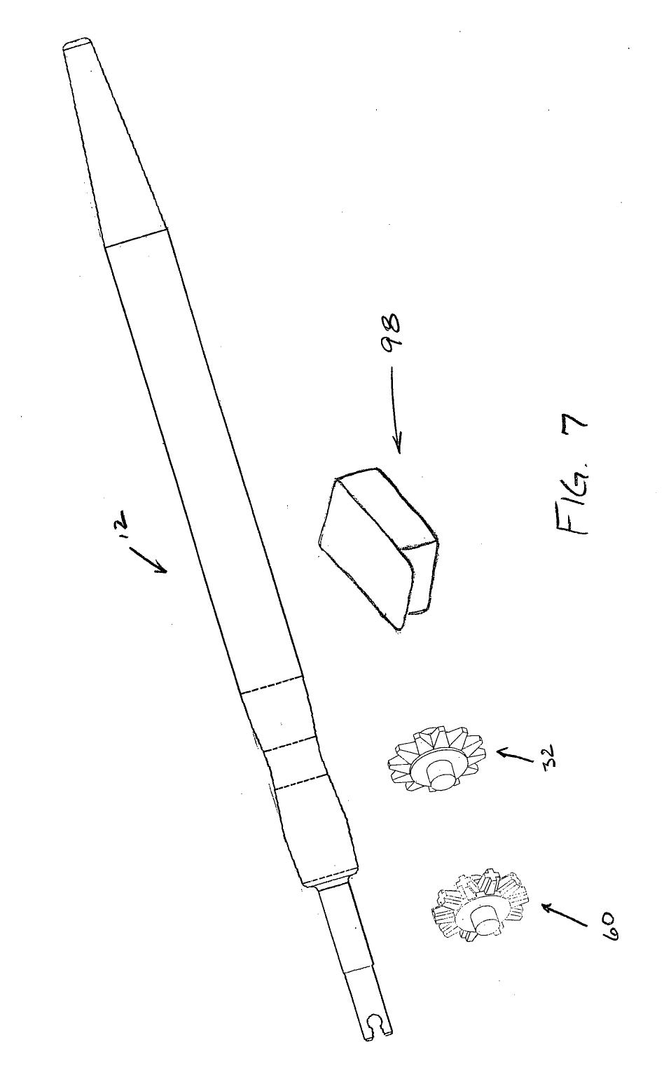

With reference to FIG. 6, another embodiment of a wheel 60 surgical marker is shown. The wheel 60 is rotatively mounted to the neck 26 between arms 28 and 30 similar to the wheel 32 shown in FIG. 5. An axle 62 of the wheel is received in the arms 28 and 30 similar to the wheel 32 in FIG. 5. The wheel 60 includes a plurality of treads 64 having markings surfaces 66. The marking surfaces 60 are cross-shaped having a first portion 68 aligned with a circumference of the wheel and a second portion 72 aligned perpendicular to the circumference of the wheel. The marking surfaces 66 are spaced from one another, similar to the marking surfaces 52 of FIG. 5. The marking surface can be flat, rounded to match the radius of the wheel, or other configuration.

When the surgeon draws the line prior to incision, the first portion 68 of the marking surfaces creates a dotted line to guide the surgeon when making the incision. The second portion 72 of the marking surface creates a hash mark substantially perpendicular to the line formed by the first portion. The hash marks created by the second portion 72 provide a guide to the surgeon when closing the incision. Accordingly, the second portions 72 are spaced apart from one another such that when a line is drawn on patient's skin, hash marks are formed a distance from one another that is conventional for the spacing of sutures, or some multiple thereof. The radius of the wheel 60 as measured to the marking surface of the tread and the angle measured between two adjacent treads is controlled by the distance at which the doctor will place sutures post-operatively and vice-versa.

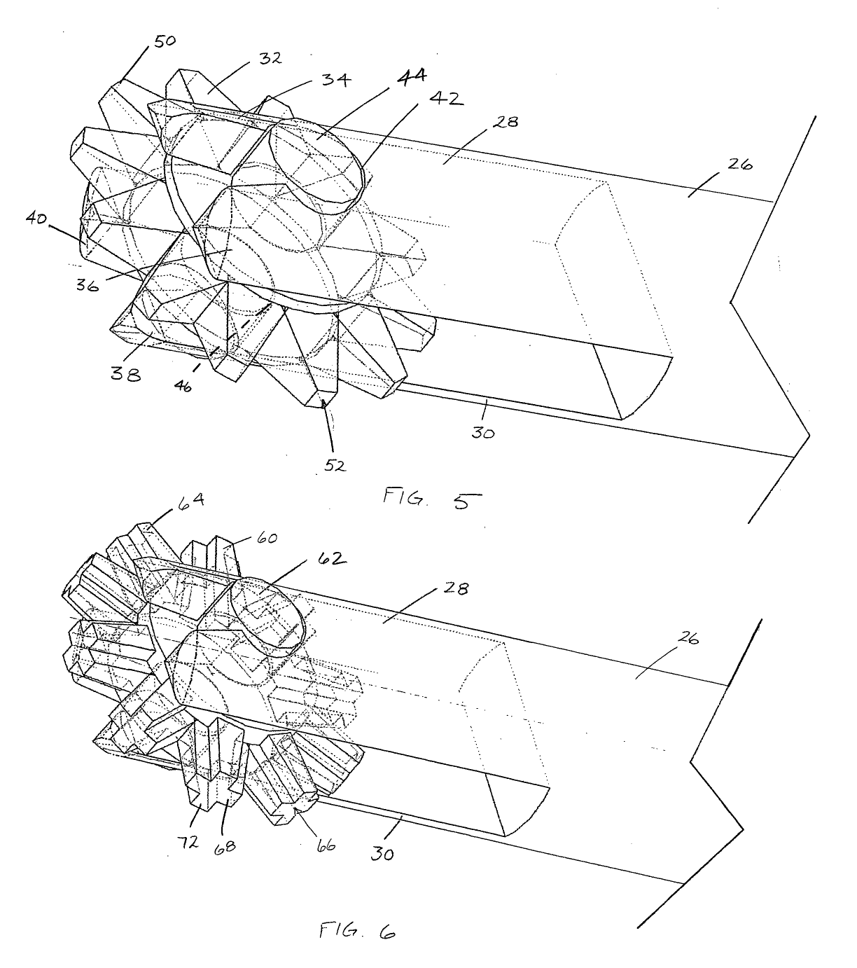

Referring now to FIG. 4, another embodiment of a surgical marker 80. The surgical marker includes a body 82 having a first end portion 84 and a second end portion 86. The body 82 or handle also includes a contoured portion 88 located in or adjacent the first end portion 84. A wheel 90, which is similar to the wheels described above, mounts to the body. The body or handle portion of the surgical marker 80 is very similar in construction to the marker 10 described above, with the exception that the body 82 includes a bend 92 where a neck 94 joins the body at the first end portion 84. The bend shown in FIG. 5 is a 30° degree bend; however, other degrees of bend are contemplated by the scope of the invention.

The bend 92 of the surgical marker 80 allows the surgeon to hold more ergonomically the surgical marker when drawing lines on the patient's skin. The bend also allows the doctor to grip the marker at an angle one typically holds a pen while writing, yet the neck portion is situated substantially normal to the patient's skin. This orientation of the wheel can lessen the likelihood of the wheel catching any of the patient's skin, which can cause unwanted bunching of the skin.

The surgical markers described above can be provided as part of a kit, see FIG. 7, that is sold as a unit. The components of the kit, e.g. the handle 12 (or other handle, for example handle 112) and the wheels 32 and 60 and nibs (not shown in FIG. 7, see FIG. 1) that attach to the handle, are all sterilized and then packaged. A plurality of wheels, some wheels having different configurations, can also be provided in each kit. Also, a plurality of nibs can be provided in each kit. A sealed container of ink 98 is also provided with each kit. The ink can be soaked into an ink pad that is packaged with a removable lid. An ink reservoir can also be provided in each kit.

A surgical marker, a method for its use and a kit that includes the marker have been described with reference to depicted embodiments. Modifications and alterations will occur to others upon reading and understanding the preceding detailed description. It is intended that the invention be construed as including all such modifications and alterations that come within the scope of the appended claims and the equivalents thereof.

What is claimed is:

1. A method of marking a patient's skin for surgery, the method comprising:
marking a patient's skin with ink using a surgical marker without penetrating the patient's skin, the surgical marker comprising a handle and a wheel rotatively attached to the handle, the wheel including a marking surface for depositing ink onto the patient's skin.

2. The method of claim 1, wherein the wheel comprises a material that selectively retains and deposits ink onto the patient's skin.

3. The method of claim 1, wherein the marking step further comprises marking the patient's skin in a manner such that locations for post-operative sutures are provided on the patient's skin.

4. The method of claim 1, wherein the marking step further comprises marking the patient's skin with an ink-soaked nib that is attached to the handle.

5. A surgical marker kit comprising:
a sterile handle having a first end portion and a second end portion;
a sterile wheel configured to attach to the first end portion of the handle, the wheel including a circumferential marking surface and comprising a material that selectively retains and dispenses ink;
ink; and
a sealed ink container for the ink, the container including a selectively removable portion to provide access to the ink.

6. The kit of claim 6, further comprising a plurality of wheels, each wheel being configured to attach to the first end portion of the handle, each wheel including a circumferential marking surface and comprising a material that selectively retains and dispenses ink.

7. The kit of claim 7, wherein at least two wheels each have a different circumferential marking surface.

8. The kit of claim 5, further comprising a nib assembly configured to attach to the second end portion of the handle.

9. The kit of claim 8, wherein the nib assembly comprises a material that selectively retains and dispenses ink.

10. The kit of claim 8, wherein the nib includes a pointed end.

11. The kit of claim 5, wherein the wheel is configured to attach to the first end portion of the handle in at least two orientations with respect to a first axis of the handle, the first axis being generally perpendicular to a rotational axis of the wheel.

12. The kit of claim 11, wherein the first axis is parallel with a central axis of the handle.

13. A surgical marker comprising:
a handle having a first end portion and a second end portion;
a wheel rotatively attached to the first end portion of the handle, the wheel comprising a flat or arcuate marking surface disposed at a peripheral edge of the wheel.

14. The marker of claim 13, wherein the wheel comprises a polymer that absorbs ink.

15. The marker of claim 13, wherein the wheel comprises a plurality of treads disposed about the peripheral edge of the wheel, each tread defining a marking surface.

16. The marker of claim 13, wherein the handle includes a removable wheel mount disposed at the first end, the wheel being rotatively attached to the wheel mount.

17. The marker of claim 16, wherein the wheel mount is configured to attach to the handle at a plurality of predetermined orientations about an axis that is generally perpendicular to an axis of rotation for the wheel.

18. The marker of claim 13, further comprising a nib attached to the second end portion of the handle.

19. The marker of claim 13, wherein the handle and the wheel are sterilized.
